(19) United States
(12) Patent Application Publication
(10) Pub. No.: US 2019/0300399 A1
(43) Pub. Date: Oct. 3, 2019

(54) Methods for Flocculating Suspended Solid Particles or Dissolved Solids Using Biobased Renewable Flocculants

(71) Applicant: The United States of America, as Represented by the Secretary of Agriculture, Washington, DC (US)

(72) Inventors: Rafael A. Garcia, Dresher, PA (US); Christine M. Nieman, Lansdale, PA (US); Matthew Essandoh, Philadelphia, PA (US)

(21) Appl. No.: 16/282,512

(22) Filed: Feb. 22, 2019

Related U.S. Application Data
(60) Provisional application No. 62/648,605, filed on Mar. 27, 2018.

(51) Int. Cl. C02F 1/56; C08L 89/00; B01D 21/01

(52) U.S. Cl. CPC C02F 1/56; C07K 14/805; B01D 21/01; C08L 89/00

(57) ABSTRACT

Disclosed are methods for aggregating suspended solid particles (e.g., kaolin) or dissolved solids in an aqueous medium, involving treating the aqueous medium with an effective amount of a flocculant (to aggregate the solids) to form aggregated solid particles or aggregated solids, and optionally separating the aggregated solid particles or aggregated solids from the aqueous medium; wherein the flocculant is a polymerized protein (e.g., polymerized hemoglobin or polymerized BSA which are proteins that have been polymerized by crosslinking).

### REFERENCE TO RELATED APPLICATION

This application claims the benefit of U.S. Provisional Application No. 62/648,605, filed 27 Mar. 2018, which is incorporated herein by reference in its entirety.

### BACKGROUND OF THE INVENTION

Disclosed are methods for aggregating suspended solid particles or dissolved solids in an aqueous medium involving mixing the aqueous medium with an effective amount of a flocculant to aggregate the solid particles or dissolved solids to form aggregated solid particles or aggregated solids, and optionally separating the aggregated solid particles or aggregated solids from the aqueous medium; wherein the flocculant is a polymerized protein.

A flocculant is a substance that causes suspended particles or dissolved solids to aggregate and form discrete flocs (Krishnan, S. V., and Y. A. Attia, Polymeric flocculants, In: Somasundaran, P., Moudgil, B. M. (Eds.), Vol. 27, Surfactant Science Series, Reagents in Mineral Technology, Marcel Dekker, Inc., New York, pp. 485-518 (1988)). Aggregation of the fine particles usually results in accelerated sedimentation to give a clarified solution. Many flocculants are polymeric, and they are used in a wide variety of processes such as wastewater clarification (Maximova, N., and O. Dahl, Curr. Opin. Colloid Int. Sci., 11: 246-266 (2006)), paper manufacture, concentration during chemical operations, and dewatering and thickening in mineral operations (Swarovsky, L., Solid-liquid separation, 4th edition, Butterworth-Heinemann, Oxford, p. 126). They are also used as filtration and centrifugation aids (Lewellyn, M. E., and P. V. Avotins, Dewatering/filtering aids, In: Somasundaran, P., Moudgil, B. M. (Eds.), Vol. 27, Surfactant Science Series, Reagents in Mineral Technology, Marcel Dekker, Inc., New York, pp. 559-578 (1988)).

Many widely-used flocculants are synthetic polymers with very high molecular weights, often in the range of 1-60 million Daltons. These flocculants can have high efficiency; however, they are non-biodegradable and are of environmental and health concern (Lee, C. H., et al., Process Safety and Environmental Protection, 92: 489-508 (2014)). The most widely used polymeric flocculant is anionic polyacrylamide (PAM) because of its high effectiveness and low toxicity to aquatic life (Nasser, M. S., and A. E. James, Effect of polyacrylamide polymers on floc size and rheological behavior of kaolinite suspensions, Colloids and Surfaces A: Physicochem. Eng. Aspects, 301: 311-322 (2007)). PAM is also applied directly to soil to prevent erosion in agricultural and construction areas (Sojka, R. E., et al., Advances in Agronomy, 92: 75-162 (2007)).

PAM is manufactured from chemicals that are made from non-renewable natural gas, and it is not rapidly degraded in the environment. Thus, renewable, biodegradable replacements are being sought. Toward this goal, derivatives of amylopectin, carboxymethylcellulose, guar gum, starch, and glycogen have been tested as flocculants (Pal, S., et al., Colloids and Surfaces A: Physiochem. Eng. Aspects, 289: 193-199 (2006)). Derivatives of chitosan have been examined as coagulation/flocculation aids in waste water treatment (Renault, F., et al., Eur. Polym. J., 45: 1337-1348 (2009)). Extracellular biopolymeric materials from microorganism fermentation have recently been investigated as a new source of renewable flocculants (Salehizadeh, H., and S. A. Shojaosadati, Biotech. Adv., 19: 371-385 (2001)). Additionally, suspensions of chitosan, starch xanthate, cellulose xanthate, and acid-hydrolyzed cellulose microfibrils have been tested for control of soil sediment runoff (Orts, W. J., et al., Industrial Crops and Products, 11: 19-29 (2000)). Known renewable flocculants and erosion control agents generally must be used at significantly higher concentrations than PAM to achieve equivalent results.

Slaughterhouse blood is an under-utilized by-product of meat production. Hemoglobin (Hb) is the most abundant blood protein and it is normally found only in the cytoplasm of red blood cells. Hemoglobin and other blood products have been shown to be a good bio-based alternative to synthetic polymer flocculants (Piazza, G. J., et al., J. Chem. Technol. Biotechnol., 2015, 90: 1419-1425 (2015); U.S. Pat. No. 8,313,654). Hemoglobin is a tetrameric molecule consisting of two alpha (a) and two beta (13) subunits with a total of 574 amino acids and a total molecular weight of ˜65 kDa. In addition, serum albumins are the most abundant proteins in blood plasma, and the second most abundant proteins in blood overall (Carter, D. C., et al., Adv. Protein Chem., 45: 153-203 (1994)). Bovine serum albumin (BSA) is also a well-known protein with a molecular weight of ˜66 kDa and which can transport different chemicals. The BSA protein consists of three homologous domains which are further sub-divided into nine loops by 17 disulfide bonds (Mandeville, J., et al., Biomacromolecules, 11(2): 465-472 (2010)). In contrast to Hb, bovine serum albumin does not have good flocculant properties.

We found that the flocculant properties of proteins can be improved (e.g., Hb) or created (e.g., BSA) through crosslinking of the proteins to form polymerized proteins.

### SUMMARY OF THE INVENTION

Disclosed are methods for aggregating suspended solid particles or dissolved solids in an aqueous medium involving mixing the aqueous medium with an effective amount of a flocculant to aggregate the solid particles or dissolved solids to form aggregated solid particles or aggregated solids, and optionally separating the aggregated solid particles or aggregated solids from the aqueous medium; wherein the flocculant is a polymerized protein.

This summary is provided to introduce a selection of concepts in a simplified form that are further described below in the detailed description. This summary is not intended to identify key features or essential features of the claimed subject matter, nor is it intended as an aid in determining the scope of the claimed subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

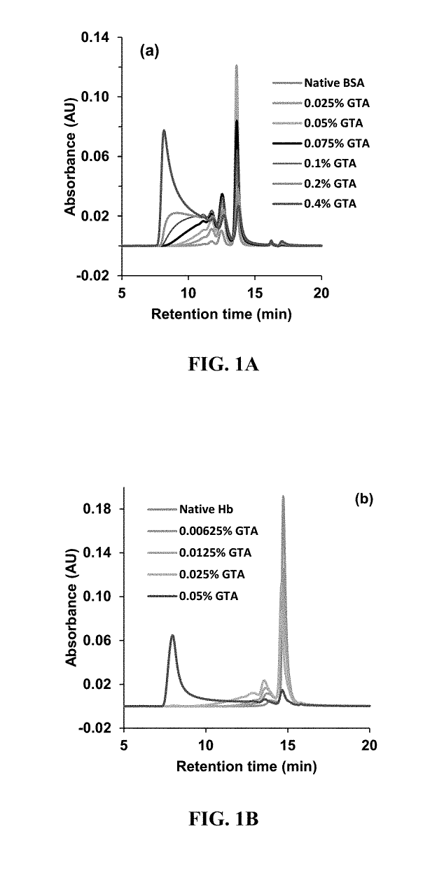

Exemplary FIG. 1A and FIG. 1B show chromatograms for size-exclusion chromatography of native and polymerized protein samples with different concentrations of glutaraldehyde (GTA) as described below. Experimental conditions used for the crosslinking include a protein concentration of 10 mg/mL, a reaction time of 2 h, and 0.1 M sodium phosphate buffer (pH 7.2) as the reaction solvent at ambient temperature. FIG. 1A is for BSA and FIG. 1B is for Hb.

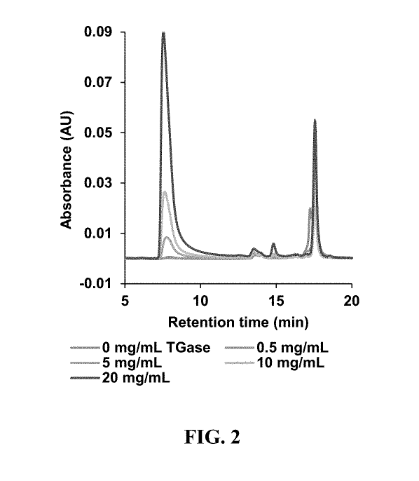

Exemplary FIG. 2 shows chromatogram for size-exclusion chromatography of BSA and polymerized BSA samples with different concentrations of transglutaminases (TGase) as described below. Experimental conditions used for the crosslinking include a protein concentration of 5 mg/mL, a reaction time of 3 h, and 0.1 M sodium phosphate buffer (pH 7.2) as the reaction solvent at ambient temperature.

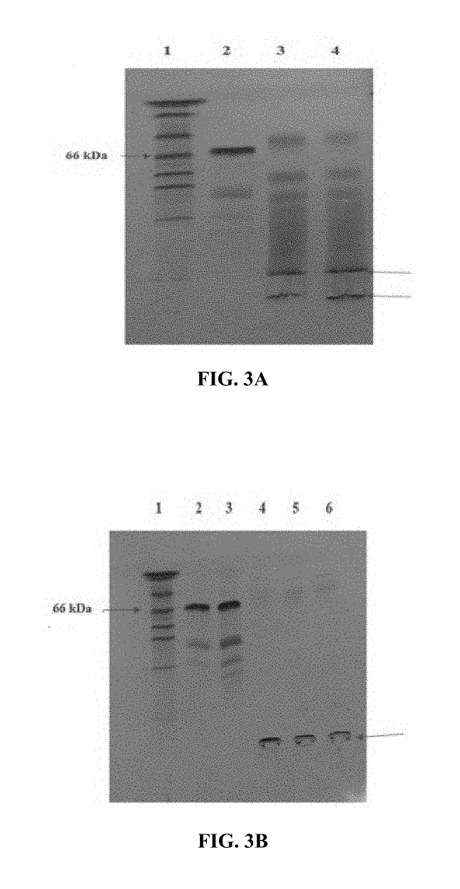

Exemplary FIG. 3A and FIG. 3B show SDS-PAGE analysis of BSA crosslinked protein as described below; FIG. 3A using different GTA concentrations and FIG. 3B using different TGase concentrations. Note: bands from top to bottom indicates lower molecular weight proteins to higher molecular weight proteins. Red arrow on the right indicates higher molecular weight protein too large to enter gel. The bands in FIG. 3A are lane 1 (protein molecular weight marker), lane 2 (BSA) and lanes 3-4 refer to the addition of 0.1% and 0.2% GTA, respectively. In FIG. 3B the bands refer to protein molecular weight marker (lane 1), lane 2 (BSA) while lanes 3-6 refer to the addition of 0, 5, 10 and 20 mg/mL TGase, respectively.

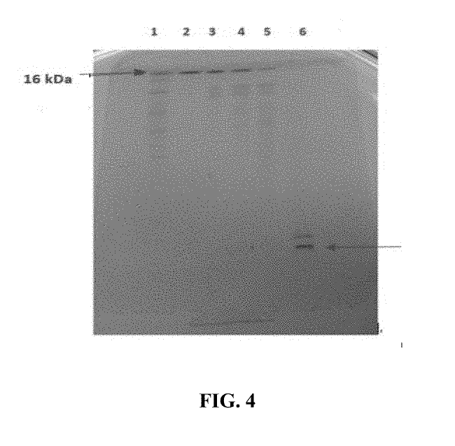

Exemplary FIG. 4 shows SDS-PAGE analysis of Hb crosslinked protein using different GTA concentration as described below. Note: bands from top to bottom indicates lower molecular weight proteins to higher molecular weight proteins. Red arrow on the right of the gel indicates polymerized protein that was too large to enter gel.

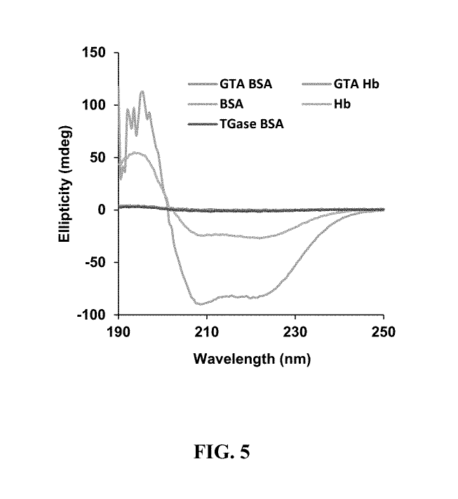

Exemplary FIG. 5 shows CD spectra of native (Hb and BSA) and crosslinked proteins (GTA-Hb, GTA-BSA, TGase-BSA) as described below. Traces for the crosslinked proteins are superimpose about 0 mdeg. Samples (0.2 mg/mL) prepared in 10 mM phosphate buffer, pH 7.2, were run at 25° C. in a 1 mm cuvette from 190 to 250 nm.

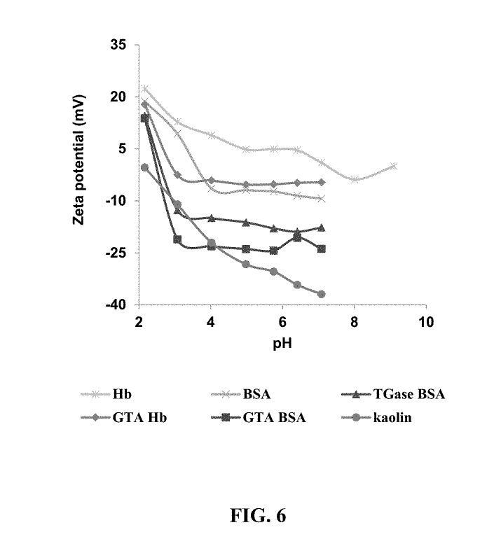

Exemplary FIG. 6 shows zeta potential as a function of pH for kaolin, native and crosslinked proteins as described below.

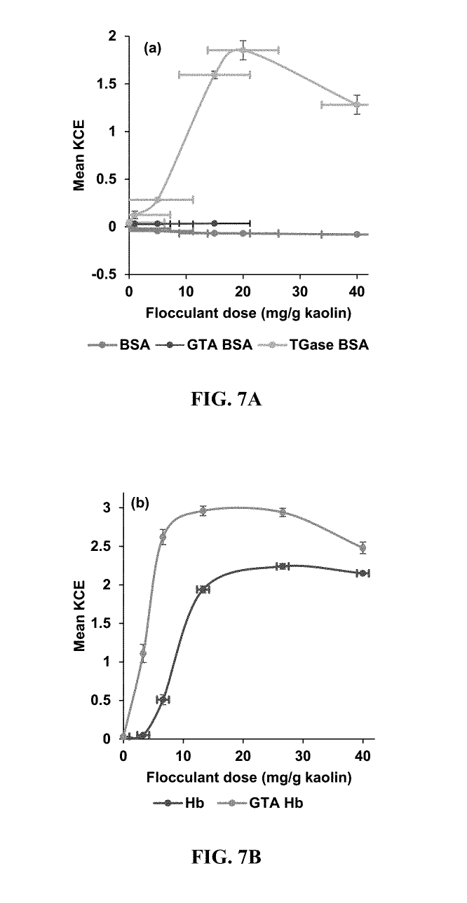

Exemplary FIG. 7A and FIG. 7B show kaolin clarification efficiency (KCE) of native and crosslinked samples as described below; FIG. 7A is for BSA and FIG. 7B is for Hb. Experimental conditions: settling time (5 h), flocculant dose (0 to 40 mg/g kaolin), temperature (20° C.), and pH (5.5). Standard deviations reported are from triplicate measurements.

### DETAILED DESCRIPTION OF THE INVENTION

Disclosed are methods for aggregating suspended solid particles (e.g., kaolin) or dissolved solids in an aqueous medium involving treating (or contacting or mixing) the aqueous medium with an effective amount of a flocculant (to aggregate the solids) to form aggregated solid particles or aggregated solids, and optionally separating the aggregated solid particles or aggregated solids from the aqueous medium; wherein the flocculant is a polymerized protein (e.g., polymerized hemoglobin or polymerized BSA which are proteins that have been polymerized by crosslinking). Polymerized protein contains multiple protein molecules linked together in a persistent manner, preferably a gel is not formed.

Crosslinking between proteins (to form polymerized proteins) can be incited by known methods including chemical crosslinkers, heat, alkaline conditions, enzymes, and photo-oxidative treatment (Gerrard, J. A., Trends Food Sci. Technol., 13(12): 391-399 (2002)). Glutaraldehyde (1,5-pentanedial) is a homobifunctional reagent with the formula CH₂(CH₂CHO)₂. Glutaraldehyde (GTA) has the capability of forming both inter- and intra-molecular protein crosslinks. This chemical has been used by various researchers to crosslink soy protein (Wang, Y., et al., J. Appl. Polym. Sci., 104(1): 130-136 (2007)), cocoa protein (Jumnongpon, R., et al., Food Chem., 134(1): 375-380 (2012)), and castor bean (Makishi, G. L. A., et al., Ind. Crops Prod., 50: 375-382 (2013)), among others. Crosslinking with GTA is common because of its high reactivity and low-cost. GTA can link the protein amino groups although the actual mechanism in which GTA crosslinks proteins is still not known (Migneault, I., et al., BioTechniques, 37: 790-802 (2004)).

Crosslinking between proteins can also be incited by enzymes. Transglutaminases (TGase) are a family of enzymes that have been found in microorganisms, mammals, and plants (Fontana, A., et al., Adv. Drug Del. Rev., 60(1): 13-28 (2008)). TGase has the ability to form both intra- and inter-molecular protein crosslinks. This enzyme initiates acyl transfer reactions between the γ-carboxamide groups of protein-bound glutamine residues acting as acyl donor and primary amines (including the amino group of lysine) as acyl acceptors. When the acyl acceptor is the ϵ-amino group of peptide-bound lysine, the resulting product is an ϵ-(γ-glutamyl) lysine crosslinked product. Recently, various researchers have employed TGase for protein crosslinking in gelled food (Grossmann, L., et al., LWT-Food Sci. Technol., 75: 271-278 (2017)), collagen, soy protein isolate, casein, and keratin (Wu, X., et al., Int. J. Biol. Macromol., 98: 292-301 (2017)), gluten mixtures and soy protein isolate (Qin, X.-S., et al., J. Sci. Food Agric., 96: 3559-3566 (2016)), and oval albumin and egg white (Ma, X., et al., Innov. Food Sci. Emerg. Technol., 29: 143-150 (2015)).

Proteins which can be crosslinked to form polymerized proteins include, for example, such proteins as hemoglobin, bovine serum albumin, soy protein, casein, gluten, and glycinin, whey protein, gelatin (hydrolyzed collagen), avenalin, and legumin.

Examples of solid materials to be flocculated include particles containing silica (e.g., clays such as kaolin), sewage solids, livestock manure, soil particles, or microorganisms such as algae cells. The solids may be negatively charged such as silica and cellulose fibers which have a negative charge (Tatsumi, D., et al., Colloids and Surfaces A: Physicochemical and Engineering Aspects, 316: 151-158 (2008)).

The flocculants of the present invention should be added to an aqueous medium containing solids (e.g., suspended solids), for which flocculation is desired, in an amount effective for that purpose. The flocculants may be used in a wide variety of processes such as wastewater clarification, paper manufacture, concentration during chemical operations, and dewatering and thickening in mineral operations.

The flocculants used in the method is an effective amount (i.e., makes possible aggregation of the solid materials). An “effective amount” or “amount effective for” is the minimum amount of the flocculant to affect the desired effect (flocculation of the solids). The precise amount needed may vary in accordance with the particular flocculant used, the solid being flocculated, and the aqueous environment in which the solid is located. The exact amount of flocculant needed can be easily determined by one of ordinary skill in the art using the teachings presented herein and using only routine experimentation. The effective amount in terms of mass flocculant per mass solids is about 5 to about 200 mg/g (e.g., 5 to 200 mg/g).

In the present method, the pH value of the aqueous medium containing suspended solids is adjusted by adding, as needed, an acid (e.g., sulfuric acid, phosphoric acid, citric acid) or acidic buffer to the aqueous media, so that the pH value of the aqueous media is about 4 to about 5.7. (e.g., 4-5.7). Although a flocculant is added to flocculate particles suspended in the aqueous medium, the flocculant cannot exhibit a good effect of flocculating the suspended particles if the pH value of the aqueous medium is not less than about 5.7 (e.g., not less than 5.7).

In addition, in the present method, either before or after the addition of the flocculant, the temperature of the aqueous medium may be adjusted to a temperature range of about 10° to about 50° C. (e.g., 10° -50° C.; preferably about 20° to about 35° C. (e.g., 20° -35° C.)). If the temperature of the aqueous medium is higher than about 75° C. (e.g., >75° C.), then the flocculant cannot satisfactorily flocculate the suspended particles since such high temperatures may cause the polymerized proteins to undergo thermal denaturation and aggregation, leading to unsatisfactory flocculant performance.

Other compounds (e.g., coagulants, co-flocculants, acids, bases or salts may be added to the composition provided they do not substantially interfere with the intended activity and efficacy of the composition; whether or not a compound interferes with activity and/or efficacy can be determined, for example, by the procedures utilized below.

“Optional” or “optionally” means that the subsequently described event or circumstance may or may not occur, and that the description includes instances in which said event or circumstance occurs and instances where it does not. For example, the phrase “optionally comprising a second flocculant” means that the composition may or may not contain a second flocculant and that this description includes compositions that contain and do not contain a second flocculant. Also, by example, the phrase “optionally adding a second flocculant” means that the method may or may not involve adding a second flocculant and that this description includes methods that involve and do not involve adding a second flocculant.

While this invention may be embodied in many different forms, there are described in detail herein specific preferred embodiments of the invention. The present disclosure is an exemplification of the principles of the invention and is not intended to limit the invention to the particular embodiments illustrated. All patents, patent applications, scientific papers, and any other referenced materials mentioned herein are incorporated by reference in their entirety. Furthermore, the invention encompasses any possible combination of some or all of the various embodiments and characteristics described herein and/or incorporated herein. In addition the invention encompasses any possible combination that also specifically excludes any one or some of the various embodiments and characteristics described herein and/or incorporated herein.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions (e.g., reaction time, temperature), percentages and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 10% to a reference quantity, level, value, or amount.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

The following examples are intended only to further illustrate the invention and are not intended to limit the scope of the invention as defined by the claims.

### EXAMPLES

Our studies utilized a protein with good flocculant properties (hemoglobin (Hb)) and one with poor flocculant properties (bovine serum albumin (BSA)), and attempted to crosslink each protein through two different methods (GTA and TGase). The cross-linked products were characterized and tested for their ability to flocculate a model suspension.

Materials: Chemicals used for the study include bovine hemoglobin (lyophilized powder), bovine serum albumin, glutaraldehyde (25% wt. in water), dithiothreitol, and protein standard mixture (15-700 kDa) which were obtained from MilliporeSigma (St. Louis, Mo.). Microbial transglutaminase was obtained from Ajinomoto USA, Inc. (Teaneck, N.J.). Water was purified to a resistance of 18 megohm-cm using a Barnstead™ E-pure™ system.

Protein crosslinking by GTA: In general, 13 mg of the protein was dissolved in 1300 μL of 100 mM sodium phosphate buffer, pH 7.2. The mixture was mixed gently. GTA was then added to a final concentration of 0.00625% to 0.4%. The reaction was then mixed briefly and left to incubate at room temperature for 2 h.

Synthesis of the enzyme catalyzed crosslinked protein: A stock solution of 5000 mg/mL of TGase solution was prepared. Enzyme crosslinking of the protein was carried out by preparing 5 mg/mL of the substrate protein using 100 mM sodium phosphate buffer, pH 7.2. The mixture was gently mixed. The final concentration of TGase varied from 0 to 20 mg/mL (0 was used as a control). The reaction was mixed gently and incubated at 40° C. for 3 h.

Degree of crosslinking: The degree of crosslinking was determined by following the work of Djoullah et al. (Djoullah, A., et al., Process Biochem., 50(8): 1284-1292 (2015)) with some slight modification. In summary, 10 mg/mL of the samples were dissolved using 2% SDS in 0.1 M phosphate buffer, pH 8. The samples were then centrifuged for 10 min at 4000× g. The preparation of o-phthaldialdehyde (OPA) reagent was carried out according to a protocol by Dinnella et al. and used immediately (Dinnella, C., et al., Food Chem., 78(3): 363-368 (2002)). Two mL of 0.80 mg/mL o-phthaldialdehyde was added to 50 μL of the supernatant taken after centrifugation. The samples were then vortexed to ensure adequate mixing before allowing them to sit at room temperature undisturbed for 5 min. The degree of crosslinking was calculated by comparing the UV absorbance at 340 nm to that of a blank solution that was prepared in the same manner without the addition of the sample.

Size Exclusion Chromatography (SEC): Samples for SEC analyses were first filtered using nylon 66 membrane (pore size=0.45 pm) before being transferred into 300 μL polypropylene (12×32 mm) snap neck vials. The HPLC instrument (Waters 2695, Waters Corporation, Milford, Mass.) was programmed to inject 15 μL of the samples using 0.05% sodium azide and 0.1 M sodium sulfate in 0.1 M phosphate buffer, pH 7.2, as the mobile phase. Samples were run for 20 min at a flow rate of 0.75 mL/min and the eluted compounds were detected at 280 nm. The column used was the TSKgel UltraSW Aggregate, 3 μm, 7.8 mm ID×30 cm (Millipore Sigma, Saint Louis, Mo.). All data generated were analyzed with Empower software (Waters Corporation).

Electrophoresis: Molecular weight determination via sodium dodecyl sulfate-polyacrylamide gel electrophoresis (SDS-PAGE) was done by mixing the crosslinked samples with sample buffer containing 10 mM Tris-HCl, 1 mM EDTA (pH 8.0), 2.5% (w/v) sodium dodecyl sulfate, 713 mM 2-mercaptoethanol and 0.1% (w/v) bromophenol blue. This mixture was heated for 5 min at 80° C. Samples were loaded onto a PhastGel gradient 4-15 gel. After the run, the gel was fixed with a 1:1 mixture of glutaraldehyde and water for 30 min, and stained with Coomassie blue for 30 min before destaining for 15 min with a 3:1:6 mixture of methanol, acetic acid, and water.

Circular dichroism analysis: Secondary structure content changes occurring after protein crosslinking was studied with circular dichroism (CD) spectrometer (Model 420, Biomedical Inc., Lakewood, N.J.). The samples were first filtered with nylon 66 membrane (pore size=0.45 μm) and run on the spectrometer at temperature 25° C., bandwidth 1 nm, path length 1 mm, and averaging time 5 s. A blank was also prepared for baseline correction. Samples were then run in the far UV range (190 to 250 nm).

Differential Scanning calorimetry (DSC): Lyophilized samples were dissolved in a degassed aqueous solution at concentration of 50-90 mg/mL. Then 750 μL was measured into the ampoules for DSC analysis using a multi-cell differential scanning calorimeter (TA Instruments, Newcastle, DE). Samples were run at 1° C./min from 20 to 120° C.

Zeta Potential Measurement: Sample buffer was prepared using nanopure water from pH 2 to 9 containing 0.2 M KCl. The pH was adjusted using dilute HCl or NaOH. About 5 mg of the sample was dissolved in 5 mL of the buffer and vortexed until well mixed. It was then incubated at room temperature for about 30 min. The supernatant obtained was transferred into a sample cell and the zeta potential analyzed using Zetasizer Nano Z (Malvern Instrument Inc., Westborough, MA). Samples were run using an equilibration time of 2 min and a temperature of 25° C.

Flocculant testing: Native and crosslinked samples were all tested as renewable flocculants. Kaolin suspension was prepared by dispersing 3 g (or 1 g in the case of BSA) of kaolin in 1 L of Malic-MES-Tris buffer (25 mM, pH 5.5). Twenty-four mL of kaolin suspension was dispensed into a glass vial and its initial turbidity measured. The amount of sample doses ranged from 0 to 40 mg protein/g kaolin. In the case of Hb and its crosslinked product, the amount of Hb used was determined from the literature (Zander, R., et al., Clin. Chim. Acta, 136(1): 83-93 (1984); Essandoh, M., et al., J. Chem. Technol. Biotechnol., 92: 2032-2037 (2017)). The amount of BSA and its crosslinked products in each dose was directly based on the dry weight of the lyophilized sample. The flocculants were then added to the suspension before shaking it for 1 min at a speed of 400 rpm. This was then followed with slower shaking at 200 rpm for 15 min. The samples were then left undisturbed in a temperature control incubator at 20°±1° C. Final turbidity of the samples was measured at 1, 3 and 5 h of incubation time. A control was also prepared and all treatments were carried out in triplicates. The results obtained from the initial and final turbidity measurement were used to calculate the kaolin clarification efficiency (KCE) (Garcia, R. A., et al., Ind. Eng. Chem. Res., 53: 880-886 (2013)).

Results and Discussion. Synthesis of crosslinked proteins using GTA: GTA was chosen because of its high reactivity and ability to generate thermally and chemically stable crosslinked proteins like collagen (Nimni, M. E., et al., J. Biomed. Mater. Res., 21(6): 741-771 (1987)). There is currently no consensus on the actual mechanism by which this GTA reaction occurs. Understanding of the reaction is complicated by the several monomeric, dimeric, and polymeric forms of GTA that can exist in solution at different temperature, pH and concentration (Migneault et al. 2004). All chemical (GTA) crosslinking reactions were carried out at room temperature using a fixed protein concentration (10 mg/mL) but different concentrations of GTA. In the case of BSA, the concentration of GTA ranged from 0.025% to 0.4%. The BSA sample turned yellow within a few minutes of reaction initiation with GTA. Other researchers also had the same observation when GTA was added to various proteins (Hopwood, D., et al., Histochem. J., 2(2): 137-150 (1970)). In the case of Hb, lower concentrations of GTA (0.00625% to 0.025%) surprisingly did not show any crosslinking as measured by the SEC, while gel formation was observed at higher concentrations (>0.1% GTA). Hopwood et al. also observed that addition of GTA to proteins can lead to gel formation which is an indication that a molecular network has formed. The concentration of GTA was therefore kept at about 0.05% to achieve Hb polymerization without gel formation.

Enzymatic crosslinking of protein using TGase: TGase from microbial origin was chosen to crosslink protein because of its calcium-independence. A simplified scheme for the reaction of TGase with proteins is shown in Scheme 1 below. The major step for this reaction was the interaction of the active site of TGase with the γ-carboxamide group of the glutamine residue of the protein to yield thioacyl-moiety (Fontana et al. 2008). The thioacyl-moiety intermediate that was formed then reacted with an amine group to form an isopeptide amide bond. In the enzymatic crosslinking reaction, protein concentration (10 mg/mL) was kept constant while the enzyme concentration was varied from 0 to 20 mg/mL. It will be shown below that the BSA-crosslinking reaction with TGase was very effective. Crosslinking of BSA was achieved as measured by SEC. However, no crosslinking was observed in the case of Hb. Other researchers have found that crosslinking Hb using TGase was not possible either in the presence or absence of dithiothreitol (de Jong, G. A., et al., J. Agric. Food. Chem., 49(7): 3389-3393 (2001)); without being bound by theory, it is possible that most of the available sites were buried and not exposed to react with the TGase enzyme.

Scheme 1. Reaction of protein with TGase to yield ϵ-(γ-glutamyl) lysine crosslink, isopeptide amide bond:

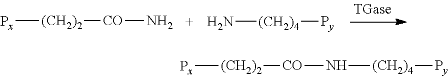

Degree of crosslinking: The formula below was used to calculate the degree of crosslinking:

$${D}_{( t )} = ( 1 - \frac{{A}_{t}}{{A}_{o}} ) \times 100 \qquad Eqn . 1$$

where D₍ₜ₎ refers to the degree of crosslinking, and Aₒ and Aₜ refer to the absorbance of the control and the absorbance of the crosslinked sample at time t, respectively. Both GTA-Hb and TGase-BSA produced a D₍ₜ₎ of ˜95%. However, GTA-BSA sample was insoluble during the degree of crosslinking experiment and therefore its D₍ₜ₎ could not be determined. Even the use of sodium dodecyl sulfate and β-mercaptoethanol to allow the unfolding of the protein and allow easy access to the amino groups of the protein did not help, an observation other researchers have also reported (Goodno, C. C., et al., Anal. Biochem., 115(1): 203-211 (1981)).

Size-exclusion chromatography: FIG. 1A displays the chromatogram obtained after crosslinking BSA with different concentrations of GTA from 0% to 0.4%. It is apparent from the chromatogram that increasing the concentration of GTA increased the molecular weight of the polymerized BSA sample. The increase in molecular weight of the polymerized protein compared to BSA was about 20- to 30-fold. Other researchers have also shown that the molecular weight of BSA increases 20-fold when subject to GTA crosslinking (Silva, C. J., et al., Food Technol. Biotechnol., 42: 51-56 (2004)).

In the case of Hb, increasing the concentration of GTA (>0.05%) led to the formation of polymerized molecules (FIG. 1B). However, concentrations that were >1% GTA gelatinized the samples and they could not be filtered. The polymerized Hb protein formed surprisingly had a high molecular mass of about 2000 kDa. As explained previously, without being bound by theory, the GTA reaction is complex, possibly utilizing many different available reaction sites on Hb. It has been reported that the reaction of Hb with GTA can give a molecular mass greater than 1000 kDa (Doyle, M. P., et al., J. Biol. Chem., 274(4): 2583-2591 (1999)).

FIG. 2 shows the chromatogram obtained when different concentrations of TGase (0-20 mg/mL) were reacted with BSA in the presence of dithiothreitol for 3 h. The samples studied showed the presence of BSA (˜13.57 min), TGase (˜14.88 min), and DTT (˜17.55 min). The use of 0.5 mg/mL TGase did not produce any crosslinking as the chromatogram obtained was similar to the control (0 mg/mL). The first appearance of polymerized BSA was observed at 5 mg/mL TGase concentration. Surprisingly, increasing the concentration of the TGase from 5 to 20 mg/mL did not alter the pattern of the chromatogram obtained except that the intensity of the polymerized sample (˜2000 kDa) formed in the reaction mixture increased.

Sodium dodecyl sulfate-polyacrylamide gel electrophoresis (SDS-PAGE): SDS-PAGE has been widely used for the separation of protein mixtures. The proteins were prepared in a denaturing and reducing environment using sodium dodecyl sulfate (SDS), heat, and β-mercaptoethanol. The proteins (SDS-bound), which had acquired a negative charge, were separated mainly based on their size. In FIG. 3A, polymerized BSA samples larger than 300 kDa can be seen (lanes 3 and 4) as shown by the red arrows on the right of the gel. In FIG. 3B, the addition of 5, 10 and 20 mg/mL TGase concentration did show crosslinking of the BSA protein in lanes 4, 5, and 6, respectively. Polymerized proteins were seen with higher molecular weight compounds (>300 kDa) that were too large to enter the gel as shown by the red arrow on the right of the gels. The SDS-PAGE results showed that TGase crosslinking of BSA protein was successful and corroborated the size-exclusion chromatography results.

SDS-PAGE analysis of native and polymerized Hb samples are shown in FIG. 4. Lanes 1 to 6 represent protein molecular weight markers, native Hb, and addition of 0.00625%, 0.0125%, 0.025% and 0.05% GTA, respectively. All samples showed the presence of ˜16 kDa protein with the exception of the 0.05% GTA sample. The ˜16 kDa band was due to the presence of monomeric Hb obtained during subunit dissociation of the tetrameric Hb in the denaturing environment. The crosslinked proteins (red arrow on the right) in lane 6 indicated Hb can be successfully polymerized using GTA.

Circular Dichroism (CD) Spectroscopy: The conformational changes on the secondary structure of native and crosslinked proteins were studied by using circular dichroism spectropolarimeter. Circular dichroism graphs obtained for the native and crosslinked samples are displayed in FIG. 5. The CD analysis was carried out in a region that is very sensitive to the secondary structural changes of protein (190 to 250 nm). Two negative bands at 222 nm and 208 nm were seen, which is typical of proteins rich in alpha helix for both BSA and Hb samples. However, upon crosslinking, the secondary structure of these native proteins were destroyed, leading to more denatured or disordered protein. Similar CD graphs were also reported for both native and modified proteins (BSA and Hb) in past studies (Varlan, A., et al., Molecules, 15(6): 3905-3919 (2010); Essandoh et al. 2017).

Differential Scanning calorimetry (DSC): Differential scanning calorimetry was carried out to study the influence of the crosslinking on the thermal stability of the proteins. Endotherm peaks were seen for both Hb and BSA (data not shown). The high denaturing temperature observed compared to what is normally seen in the literature might, without being bound by theory, be due to the higher concentrations (90 mg/mL) of the samples used for the analysis. Other researchers have also found the denaturing temperature of BSA increases with increasing concentration of sample used (Michnik, A., J. Therm. Anal. Calorim., 71(2): 509-519 (2003)). Of note was the broader nature of the BSA peak that started to denature around 65° C.; the BSA peak was broad compared to the Hb peak which was narrow. None of the crosslinked samples showed any sign of denaturing under the range of temperatures studied. Without being bound by theory, this might be due to the fact that the protein might already have been denatured.

Zeta Potential Measurement: At the isoelectric point, the total number of positive charges from protein amino groups and negative charges from the carboxylic groups are equivalent. At low pH (less than the isoelectric point of the protein) the protein is positively charged while at high pH (greater than the isoelectric point of the protein) the protein is negatively charged. Zeta potential results are shown in FIG. 6. The isoelectric point of Hb, GTA-Hb, BSA, GTA-BSA, TGase-BSA, and kaolin were 7.3, 3.0, 3.6, 2.5, 2.7 and 2.1 respectively. After crosslinking, the isoelectric point of both proteins decreased. This was as a result of the reduction or utilization of the free amino groups during crosslinking. For example, highly positively charged amino groups are converted to isopeptide amide bonds during crosslinking. It will be shown below how these zeta potential results were surprisingly crucial when it comes to the flocculating ability of the native and crosslinked proteins.

Application of crosslinked proteins as bioflocculant: The ability of the crosslinked samples to be used as bioflocculants was tested. The results of the flocculation studies are presented in FIG. 7A and FIG. 7B. Both the BSA and crosslinked GTA-BSA samples show little to no flocculation activity (FIG. 7A); the BSA (blue) and GTA-BSA (red) samples did not show any clarification as their mean KCE values (y-axis) were around zero. Without being bound by theory, it is possible that BSA is not an active flocculant primarily because of its low isoelectric point (pI=3.6). Other researchers have also shown that BSA is not a good flocculant (Piazza, G. J., et al., Appl. Biochem. Biotechnol., 166: 1203-1214 (2012)). Even after GTA crosslinking (GTA-BSA), there was surprisingly no enhancement in its flocculation activity. Thus, under this entire study, there was electrostatic repulsion between the negatively charged BSA (or GTA-BSA) with the negatively charged kaolin particles (pI=2.1). The observed flocculation activity surprisingly exhibited by TGase-BSA (KCE=1.85) may, without being bound by theory, be due to bridging mechanism, which will be explained below.

In the case of native Hb (peak KCE=2.24), clarification of kaolin suspension was observed (FIG. 7B); the native Hb or Hb (purple) resulted in flocculating activity with a mean KCE values (y-axis) around 2.2, thus the particles in the suspension were able to settle out of the solution. At pH 5.5, native Hb (pI=7.3) is positively charged while kaolin is negatively charged. There was, therefore, an electrostatic interaction between Hb and the kaolin particles. Thus the Hb flocculation mechanism utilized here was charge neutralization. Recently, charge neutralization was also realized to be the main flocculation mechanism employed by native and methylated Hb (Essandoh et al. 2017).

However, it is surprising that in the case of the crosslinked GTA-Hb (KCE=2.96) that there was substantial increase in its clarification efficiency despite its low isoelectric point (pI=3.0). Thus charge neutralization cannot be used to explain the highly enhanced flocculation ability of the high molecular weight crosslinked GTA-Hb. Without being bound by theory, although GTA-Hb carries an overall negative charge at pH 5.5, different portions of the molecule can have local charges that are either positive or negative. A positively charged portion of GTA-Hb can be electrostatically attracted to the negatively charged kaolin particle surface. A very large molecule such as GTA-Hb (˜2000 kDa) can plausibly be electrostatically attached to two or more kaolin particles simultaneously, effectively tethering the particles together and promoting flocculation. This flocculation mechanism is known in the literature as ‘bridging’ and is used to explain the observed phenomenon of negatively charged particles being flocculated by negatively charged flocculants.

Conclusions: Hb and BSA proteins were successfully polymerized using both enzymatic and chemical (e.g., GTA) means. Electrophoresis (or size exclusion chromatography), zeta potential measurement, and differential scanning calorimetry studies confirmed that these polymerized samples exhibited high molecular weight (˜2000 kDa), low isoelectric points, and were thermally stable (>120° C.). We showed that polymerized protein samples can surprisingly be used as an effective bioflocculant, which is important considering the fact that commercial polymeric flocculants are non-biodegradable coupled with their environmental and ecological concerns.

All of the references cited herein, including U.S. Patents and U.S. Patent Application Publications, are incorporated by reference in their entirety. Also incorporated by reference in their entirety are the following references: Lee, C. S., et al., Process Saf. Environ. Prot., 92(6): 489-508 (2014); Li, H., et al., AlChE J., 55(8): 2070-2080 (2009); Tang, W., et al., PloS one, 9(12): e114591 (2014); Zhu, L., et al., IUBMB Life, 43(1): 207-216 (1997)).

Thus, in view of the above, there is described (in part) the following:

A method for aggregating suspended solid particles or dissolved solids in an aqueous medium, said method comprising (or consisting essentially of or consisting of) treating said aqueous medium with an effective amount of a flocculant to aggregate said solid particles to form aggregated solid particles or to aggregate said dissolved solids to form aggregated solids or said aggregated solids, and optionally separating said aggregated solid particles from said aqueous medium; wherein said flocculant is a polymerized protein.

The above method, wherein said polymerized protein is polymerized hemoglobin; wherein said polymerized hemoglobin is hemoglobin in animal blood where the hemoglobin has been polymerized or wherein said polymerized hemoglobin is hemoglobin isolated from animal blood where the hemoglobin has been polymerized.

The above method, wherein said polymerized protein is bovine serum albumin.

The above method, wherein said animal blood is from agricultural livestock. The method wherein said agricultural livestock is selected from the group consisting of poultry, pigs, sheep, or cattle. The method wherein said poultry are chickens or turkeys.

The above method, wherein said method comprises (or consists essentially of or consists of) treating said aqueous medium with an effective amount of a flocculant to aggregate said solid particles or solids to form aggregated solid particles or aggregated solids, and separating said aggregated solid particles or aggregated solids from said aqueous medium.

The above method, wherein said method comprises (or consists essentially of or consists of) treating said aqueous medium with an effective amount of a flocculant to aggregate said solid particles or solids to form aggregated solid particles or aggregated solids, and separating said aggregated solid particles or aggregated solids from said aqueous medium by gravity settling, centrifugation, filtration, or dissolved air floatation.

The above method, wherein said flocculant has a molecular weight of at least about 200 kDa (up to about 12 MDa).

The above method, wherein said flocculant has a molecular weight of about 200 to about 4000 kDa (e.g., 200 to 4000 kDa).

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein).

The invention illustratively disclosed herein suitably may be practiced in the absence of any element (e.g., method (or process) steps or composition components) which is not specifically disclosed herein. Thus the specification includes disclosure by silence (“Negative Limitations In Patent Claims,” AIPLA Quarterly Journal, Tom Brody, 41(1): 46-47 (2013): “ . . . Written support for a negative limitation may also be argued through the absence of the excluded element in the specification, known as disclosure by silence . . . Silence in the specification may be used to establish written description support for a negative limitation. As an example, in Ex parte Lin [No. 2009-0486, at 2, 6 (B.P.A.I. May 7, 2009)] the negative limitation was added by amendment . . . In other words, the inventor argued an example that passively complied with the requirements of the negative limitation . . . was sufficient to provide support . . . This case shows that written description support for a negative limitation can be found by one or more disclosures of an embodiment that obeys what is required by the negative limitation . . . .”

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

We claim:

1. A method for aggregating suspended solid particles or dissolved solids in an aqueous medium, said method comprising treating said aqueous medium with an effective amount of a flocculant to aggregate said solid particles to form aggregated solid particles or to aggregate said dissolved solids to form aggregated solids, and optionally separating said aggregated solid particles or said aggregated solids from said aqueous medium; wherein said flocculant is a polymerized protein.

2. The method according to claim 1, wherein said polymerized protein is polymerized hemoglobin; wherein said polymerized hemoglobin is hemoglobin in animal blood where the hemoglobin has been polymerized or wherein said polymerized hemoglobin is hemoglobin isolated from animal blood where the hemoglobin has been polymerized.

3. The method according to claim 1, wherein said polymerized protein is bovine serum albumin.

4. The method according to claim 2, wherein said animal blood is from agricultural livestock.

5. The method according to claim 4, wherein said agricultural livestock is selected from the group consisting of poultry, pigs, sheep, or cattle.

6. The method according to claim 5, wherein said poultry are chickens or turkeys.

7. The method according to claim 1, wherein said method comprises treating said aqueous medium with an effective amount of a flocculant to aggregate said solid particles or solids to form aggregated solid particles or aggregated solids, and separating said aggregated solid particles or aggregated solids from said aqueous medium.

8. The method according to claim 1, wherein said method comprises treating said aqueous medium with an effective amount of a flocculant to aggregate said solid particles or solids to form aggregated solid particles or aggregated solids, and separating said aggregated solid particles or aggregated solids from said aqueous medium by gravity settling, centrifugation, filtration, or dissolved air floatation.

9. The method according to claim 1, wherein said flocculant has a molecular weight of at least about 200 kDa.

10. The method according to claim 1, wherein said flocculant has a molecular weight of about 200 to about 4000 kDa.
